(19) United States
(12) Patent Application Publication
(10) Pub. No.: US 2005/0123917 A1
(43) Pub. Date: Jun. 9, 2005

(54) Method for characterising and/or identifying active mechanisms of antimicrobial test substances

(75) Inventors: Harald Labischinski, Wuppertal (DE); Guido Schiffer, Velbert-Langenberg (DE); Jurgen Schmitt, Heidelberg (DE); Thomas Udelhoven, Trier (DE)

Correspondence Address: FOLEY AND LARDNER;SUITE 500, 3000 K STREET NW, WASHINGTON, DC, 20007, US

(73) Assignee: Synthon KG

(21) Appl. No.: 10/495,042

(22) PCT Filed: Nov. 12, 2002
(86) PCT No.: PCT/EP02/12642

(30) Foreign Application Priority Data
Nov. 12, 2001 (DE) 101 55 185.1

(51) Int. Cl. C12Q 1/68; G06F 19/00; G01N 33/48; G01N 33/50

(52) U.S. Cl. 435/6; 702/19

(57) ABSTRACT

The invention relates to a method for the characterisation and/or identification of active mechanisms of antibacterial test substances, by means of IR (infrared) analyses, FT-IR (Fourier Transform Infrared) analyses, Raman analyses, or FT-Raman (Fourier Transform Raman) analyses.

The invention concerns a process for the characterisation and/or identification of mode of action mechanisms of in particular antimicrobially acting test substances with the aid of IR (infrared), FT-IR (Fourier-Transform infrared), Raman or FT-Raman (Fourier-Transform Raman) analyses.

Epidemiological studies confirm that the resistance rates of pathogenic micro-organisms, such as bacterial germs, against normal inhibitors, such as antibiotics, antimycotics and other chemotherapeutic agents, have increased continually over the course of the last two decades [Levy S. B. (2001) Antibiotic resistance: consequences of inaction. Clin. Infect. Dis. Sep. 15; 33 Suppl. 3: pp. 124-9]. In order to ensure the possibility of future treatment of bacterial infections under these circumstances of increasing resistance rates against known antibiotics, great efforts are being undertaken throughout the world to develop and identify new leading structures of antibiotics therapy. In this respect, the investigation of the action mechanism of such new lead-structures is of central importance for the research and development of antimicrobial substances. The term action mechanism (target identification) refers to the identification of the metabolism pathways down to the level of individual molecular processes which have a causal connection with the antimicrobial effect of a new leading structure. On the one hand, the knowledge of the microbial target structure enables the rapid and efficient optimisation of the leading structure in vitro, e.g. in a sub-cellular target assay; on the other hand, potential toxicological side effects based on the inhibition of a homologous target possibly also present in the host can be recognised at an early stage by means of a relevant comparison test. With the knowledge of the molecular target or target area, it is also possible to obviate the development of an antimicrobial test substance with a non-selective action mechanism (e.g. general membrane-destroying detergence effect, destruction of the membrane potential by ionophores, intercalation in nucleic acids), which amongst other things can also save research costs.

Modern antibiotics currently used in human therapy are characterised by their specific effect on a metabolism process essential to the survival of the bacterium (see for example Graefe U. (1992) Biochemie der Antibiotika, pp. 15-39, Spektrum Akademischer Verlag, Heidelberg, Berlin, N.Y.). The overwhelming number of classes of antibiotics known to date inhibit or deregulate the biosynthesis of bacterial macro-molecules such as DNA (examples: Chinolone, Novobiocin), RNA (examples: Rifampicin, Streptolydigin, Lipiarmycin, Holomycin), protein (examples: Macrolide/Ketolide, Aminoglycoside, Tetracycline, Oxazolidinone) or Peptidoglycan (examples: β-Lactame, Fosfomycin, Vancomycin, Moenomycin). Other antibiotics exert their effect by inhibiting the metabolism pathways of the intermediary metabolism (e.g. Sulfonamide and Trimethoprim as inhibitors of the C₁ metabolism; Cerulenin as an inhibitor of fatty acid biosynthesis).

The antibiotic effect can frequently be traced back directly to the inhibition of a defined enzyme or enzyme family; for example, β-Lactames irreversibly inhibit the enzyme family of Penicillin-binding proteins essential for cell wall synthesis, and thus ultimately induce autolysis of the bacteria cell. In other cases, larger, macro-molecular structures, such as Ribosomes—ribonucleic protein complexes that catalyse the translation of mRNA into a protein sequence—serve as the point of attack of antibiotics (e.g. Macrolide) (Graefe U. (1992) Biochemie der Antibiotika, pp. 15-39, Spektrum Akademischer Verlag,

Heidelberg, Berlin, N.Y., Russell A. D., Chopra I. (1996) Understanding Microbial Action and Resistance, 2^{nd} Edition, pp. 28-83, Ellis Horwood, London).

According to the state of the technology so far, the following methods in particular are used, either individually or in combination, for the investigation of the action mechanism of antimicrobially active substances:
1) In the mortification experiment (Rybak M. J. et al. (2000) In vitro activities of daptomycin, vancomycin, linezolid, and quinupristin-dalfopristin against Staphylococci and Enterococci, including vancomycin-intermediate and -resistant strains. Antimicrob. Agents Chemother. 44(4) 1062-1066), the number of surviving bacteria is determined in relation to the acting time of the substance to be tested in comparison to an untreated control culture under otherwise equivalent growth conditions. This however allows only a rough distinction to be made between bacteriostatic (growth-inhibiting) and bacteriocidal (mortifying) substances.
2) In the metabolite introduction test (Oliva B. et al. (2001) antimicrobial properties and mode of action of the pyrothine holomycin. Antimicrob. Agents Chemother. 45, pp. 532-539), bacterial cells are incubated under suitable conditions in the presence of the leading structure to be tested with such radioactive preliminary stages for important metabolism pathways (e.g. [¹⁴C]-Thymidin, [¹⁴C]-Uridin, [¹⁴C]-Leucin, [¹⁴C]-N-Acetylglucosamin), which are selectively introduced into high-molecular materials precipitable with acids or organic solvents (DNA, RNA, protein, Peptidoglycan). Following separation of the high-molecular from the low-molecular soluble fraction of the radioactivity by filtration or centrifugation, the radioactivity in the high-molecular fraction represents a measure of the synthesis performance of the cell in the relevant metabolic pathway. This test can be automated (Renick P. J. and Morris T. W. (2000) Simultaneous parallel assays for inhibition of major metabolic pathways in intact cells of Staphylococcus aureus. Poster F-2023 Session 211, 40^{th} Interscience Conference on Antibacterial Agents and Chemotherapy, Toronto), although it only identified targets in he range of the macro-molecular synthesis. Targets in the range of the intermediary metabolism are generally not identified. A further limitation on the process is its reliance on the availability of radioactively marked selective preliminary stages.
3) In the case of the genetic methods (Zhang L. et al. (2000) Regulated gene expression in Staphylococcus aureus for identifying conditional lethal phenotypes and antibiotic mode of action. Gene 255(2): 297-305), the construction generally used is that of supra- or sub-expression mutations (individual strains or mutant libraries), which frequently lead to a change in the sensitivity toward the leading structure to be tested, insofar as the mutation concerns a gene of the metabolic pathway concerned. A further procedure consists of the selection of mutants that are resistant to the test substance. A vector library can be produced from the genomic DNA of these mutants, on the basis of such substances as plasmides, cosmides and bacteriophages amongst others.
   With the aid of current molecular-genetic and microbiological techniques, it is possible to identify the mutation, and thus delimit the potential target or identify a gene having some relationship to the target. These methods are very labour-intensive, cannot be automated, and thus very costly in terms of time and resources.
4) Binding experiments for the. direct confirmation of the binding of the leading structure to be tested to its target frequently give very direct indications of the action mechanism (Spratt B. G. (1977) Properties of penicillin-binding proteins of Escherichia coli K12. Eur. J. Biochem. 72: 341-352). These make use of the fact that the reciprocal effects between antimicrobially active substances and their sites of action are as a rule very strong (e.g. covalent bonding in the base of β-Lactames), and therefore frequently withstand analytical manipulation aimed at isolation and detection of the target inhibitor complex. However, the disadvantage is that as a rule, suitably (e.g. radioactively) marked inhibitors must be available, which can often not be obtained, or if so only at considerable cost, and in the case of weaker, non-covalent interactions in particular, the required complex cannot be isolated. Added to this is the fact that a newly modified procedure must be established for every individual case (e.g. depending on the sub-cellular location of the target, and the nature of the binding between target and inhibitor).

The described methods of target characterisation have the disadvantage that in the case of a mortification experiment, they provide only a small information content or are generally and uniformly applicable to different target areas, and in addition also take up a great deal of time. The investigation of the action mechanism can extend in individual cases over several years. Even 14 years after the first description of daptomycin (Allen N. E. et al. (1987) Inhibition of peptidoglycan biosynthesis in gram-positive bacteria by LY146032. Antimicrob. Agents Chemother. 31, 1093-1099), the molecular action mechanism has still not been completely clarified.

In addition to the mentioned methods of target identification, the initial approaches have also been described for the use of physical measurement techniques, such as FT-IR spectroscopy, in the characterisation of the action mechanism of antibacterially acting substances (Naumann D. et al. (1991) The characterization of microorganisms by Fouriertransform infrared spectroscopy (FT-IR). In: Modem techniques for rapid microbiological analysis, Nelson W. H., VCH, pp. 43-96, Weinheim; EP 0 457 780 B1). The principle of this procedure consists of the spectroscopic confirmation of the change in the molecular composition caused by the incubation of the bacteria cell with the test substance in comparison to an untreated control culture. This procedure is based on an evaluation of bands in the form of area integrals, which are compared with one another. This is used for interpretation purposes in cases where molecular changes occur in the cell, without being able to deduce from this any typical pattern of action. Although the procedure is reproducible, it is neither generally or uniformly applicable in the form described, nor can it be automated. For instance, new action mechanisms, for which no inhibitors are as yet available as reference compounds, cannot be analysed. Depending on the action mechanism, the process also requires various time-consuming analyses.

The task of the invention is based on developing a procedure for the characterisation and/or identification of action mechanisms of antimicrobial test substances. The procedure described by the invention should be quick, should enable a uniform characterisation and/or identification of different action mechanisms, and should, by means of its capability of automation, also be able to be used effectively both in industrial research and development and in routine laboratory work.

This task is solved by the procedure described by the invention, which contains the following steps:
a) Treatment of a microbial culture with the test substance;
b) Recording of at least one spectrum (test spectrum) from the group of IR, FT-IR, Raman and FT-Raman spectra;
c) Comparison of the test spectrum/spectra from b) with one or more spectra (reference spectra), divided into one, two or more classes, of microbial cultures treated with reference substances;
d) Allocation of the test spectra to one, two or more of the classes of reference spectra in the reference database.

In the preferred version of the invention described, the comparison is carried out by means of mathematical processes of pattern recognition.

In a further preferred version of the invention described, the reference spectra and/or test spectra are processed in such a way as to allow the automatic recognition of the characteristic spectral changes and patterns.

In a further preferred version of the invention described, the classification is carried out by means of a pattern recognition system that can distinguish between two or more classes simultaneously.

In a further preferred version of the invention described, the class specific information of a spectral pattern is stored in a classification model or by means of weights in an artificial neural network.

In a further preferred version of the invention described, the comparison of the test spectra with the reference spectra is carried out by means of the classification model.

The functional groups of all biochemical components of a microbial culture, such as peptides, proteins, polysaccharides, phospholipids, nucleic acids and intermediary metabolites, all contribute to the spectrum of this culture, and produce a specific, biochemical fingerprint. Due to their large number of components, these spectra have a very complex composition, and reflect many different vibration modes of the biomolecules of the cell wall, the cytoplasm membrane, the cytoplasm itself and the extra-cellular polymer substances (e.g. Peptiodglycan, lipopolysaccharide, (lipo)-teichon acids). Despite their complexity, the spectra are very specific of the composition, properties or condition of a microbial culture, which should preferably be a pure microbial culture. Since the composition, condition and properties of microbial cultures change in a specific way under the effect of treatment with antimicrobial substances, depending on the substance used, the spectroscopic recording of these changes can be used for the identification and/or characterisation of the action mechanism involved. These action mechanisms may for example include inhibitors of the protein biosynthesis, the RNA or DNA metabolism, the cell wall or lipid metabolism, membrano-trophic substances or DNA intercalators. The action mechanisms referred to are examples only, and are by no means exhaustive, and more could easily be added by any specialist in the field.

The procedure described by the invention combines the advantages of spectroscopic measurement technology with a dedicated mathematical evaluation of the information content of spectra.

The reference database is built up by treating microbial cultures with test substances whose action mechanism is known with identical parameters of cultivation conditions such as temperature, pH-level, cultivation medium and time. Reference spectra of the microbial cultures treated in this way are then recorded, and added to the database, allocated to the class belonging to the relevant action mechanism.

The reference spectra allocated to a class show an identical or similar structure in one or more of the selected wavelength ranges, which differs significantly from the structure of the reference spectra of other classes in the selected wavelength ranges.

The selection of the wavelength ranges used for the differentiation of the classes (“feature selection”) can be made by means of multi-variate statistical procedures, such as variance analysis, co-variance analysis, factor analysis, statistical distance dimensions such as the Euclidian distance or the Mahalanobis distance, or. a combination of these methods together with an optimisation process such as genetic algorithms.

An automated and optimised search for wavelengths can be performed through the use or combination of genetic algorithms. In this way, the wavelengths can be compiled into a ranking more quickly and efficiently, in the best way possible for the classification. The main feature here is that an automated identification is performed of the spectral changes which make a contribution to the spectral change. These identified ranges can be used in order to build up an automated classification system. The evaluation is ideally made through a combination of genetic algorithms with the co-variance analysis.

Prior to the wavelength selection, preliminary processing of the reference spectra can be carried out in order to increase the spectral. contrast by means of the formation of derivations, deconvolution, filtering, noise suppression or data reduction by wavelet transformation or factor analysis.

The allocation of the reference spectra into the different classes is carried out by means of mathematical classification methods such as multi-variate, statistical processes of pattern recognition, neuronal networks, methods of case-based classification or machine learning, genetic algorithms or methods of evolutionary programming. Several synthetic neuronal networks can be used as a feed-forward network with three layers and a gradient decent method as the learning algorithm. The classification system may show a tree structure, in which classification tasks are broken down into partial tasks, and the individual classification systems in a unit are combined to form a hierarchical classification system, in which all stages of the hierarchy are processed automatically during the course of the evaluation. The individual stages of the classification systems may take the form of neuronal networks, which have been optimised for special tasks.

A combination of neuronal networks with a genetic algorithm is also possible to undertake an optimisation of the classification through neuronal networks. This optimisation can for example be carried out by improvement of the network architecture or the learning algorithm.

The reference database can also take the form of a synthetic neuronal network, in which the spectral information is stored in the form of neuronal weights, and can be sued in the evaluation.

The creation of the reference database for the characterisation and/or identification of the action mechanisms in a microbial culture fundamentally need be carried out only once. There also exists the facility of extending the database at any time. This can be done, for example, by adding further substances to the classes already contained in the database. Apart from this, the reference database can also be extended to include other action mechanisms not so far contained in the database. In such cases, the database must be re-organised as described above, whereby the spectral data records already used for the creation of the previous database do not need to be re-created as long as the microorganism used, its culture conditions and the spectral measurement parameters are not changed.

The allocation of a test spectrum to one, two or more classes of reference spectra can be made by means of mathematical classification methods based on pattern recognition. Methods that enable simultaneous classification into several classes, such as is the case with classification by means of synthetic neuronal networks, are particularly suitable for the automated and efficient classification of several classes. Processes based on the probability density function, the correlation matrix, methods of case-based classification or machine learning, genetic algorithms or methods of evolutionary programming are also suitable in principle. The classification system may consist of several sub-units with a tree structure, in which classification tasks are broken down into partial tasks, and the individual classification systems in a unit are combined to form a hierarchical classification system, in which all stages of the hierarchy are processed automatically during the course of the evaluation.

The test spectrum of a substance with an unknown action mechanism is obtained with exactly the same culture(s) (identical micro-organism strains) that are also used for the recording of the reference data. All culture conditions (such as temperature, pH-level, cultivation medium and time) must also correspond exactly to those maintained during the creation of the reference database.

The allocation of a test spectrum to one, two or more classes of reference spectra is carried out by means of mathematical classification methods such as multi-variate, statistical processes of pattern recognition, neuronal networks, methods of case-based classification or machine learning, genetic algorithms or methods of evolutionary programming.

The treatment of the microbial culture prior to recording of the spectra can be carried out as follows:

The microorganisms (test germs) are cultivated in a suitable, microbiological nutrient medium, which may be liquid or solid. The test substance or reference substance is then brought into contact with the bacteria. At the end of a suitable acting time, which should preferably be between five and 500 minutes, the treated bacteria are separated from the test substance or reference substance, e.g. by centrifugation or filtration if carrying out the procedure using a liquid culture, or by removing the cells from a solid nutrient medium with the aid of a hypodermic. In order to remove residues of the test preparation, the cells are washed once, or preferably several times, in a suitable volume.

The spectra can then be recorded. The steps of filtration or centrifugation can also be circumvented by carrying out a measurement of test germs with the test substance in comparison to an untreated control sample of the test germs. An automated subtraction of the spectra must then be performed. The resulting spectrum obtained is therefore based only on the changes caused by the active substance.

The procedure described by the invention can be performed equally well with IR, FT-IR, Raman and FT-Raman spectra.

The recording of IR spectra is typically performed in the spectral range of the so-called medium infrared, between 500-4,000 cm⁻¹, although it can also be measured in the near infrared range between 4,000 and 10,000 cm⁻¹ or extended to include this range.

Any of the known spectroscopic measurement arrangements can be used for the recording of IR or Raman spectra, such as transmission/absorption, weakened total reflection, direct or diffuse reflection or IR fibre-optic technique. The preferred method is measurement by transmission/ absorption.

The samples of the microbial culture are preferably either solid or liquid. The measurement is best carried out with the aid of multi-cuvettes for the measurement of several samples or the use of micro-spectrometric techniques. These include FT-IR, Raman and FT-Raman microscopy or other processes of beam focussing. This allows the number of samples to be reduced to a minimum and the use of an automated sample preparation and measurement procedure, in order to increase the sample throughput and establish a level for high-throughput screening. Sample carriers, as used for micro-titration plates, or throughflow cuvettes can also be used. The use of throughflow cuvettes, coupled with an automated HPLC sample delivery system, would also enable an increased sample throughput. Infrared fibre-optics can also be used for automation of the measurement process more independent of-the location.

All water-insoluble optical materials commonly used in IR spectroscopy can be used as materials for cuvettes or sample carriers for the preparation variants described above, such as Ge, ZnSe, CaF₂, BaF₂, although ZnSe has proven very suitable as a multi-sample element. Keyed metal plates or micro-metal grills are also suitable as sample holders, particularly if they are designed to the same scale as the micro-titration plates for a large number of samples, and as disposable materials.

The sample volume for the recording of IR spectra can be kept very small, and need only be a few μl (2-5 μl). Depending on the given conditions with or without beam focussing, substance quantities in the jg-ng range can be used. The diameter of the sample areas illuminated varies between 1-6 mm and 5-50 μm with micro-focussing.

In the case of Raman measurements, another possibility is measurement in a liquid culture, which can be carried out direct in the sample preparation vessels, e.g. micro-titration plates. This can offer a considerable time benefit coupled with a high degree of automation, since the processing times are reduced and sample preparation steps can be omitted. The optimum positioning of the Raman signal can be achieved by the use of confocal beam guidance, in order to eliminate interference signals and improve the signal-to-noise ratio. An arrangement of simultaneously used light sources or the corresponding replication of the stimulating beam and direction onto the sample for the Raman measurement, and the use of detectors (e.g. CCDs) arranged in parallel, can also significantly increase the sample throughput and the automation capability.

The test substance may be an inhibiting agent. The concentration of inhibiting agent with which the bacterial culture is treated should preferably be in the range of 0.1× to 20× the minimum inhibiting agent concentration (MIC) for the test substance. The minimum inhibiting agent concentration is the minimum concentration of an antibiotic which inhibits the growth of a test germ over a period of 18-24 hours. The inhibiting agent concentration can therefore be determined according to standard microbiological procedures (see for example The-National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000.). The test spectra are recorded from a microbial culture that has been treated with the inhibiting agent in one, or preferably in several concentrations.

The procedure described by the invention is suitable for the examination of a wide range of cell cultures. A preferred group of cell cultures consists of microbial cell cultures such as bacteria, moulds, yeasts, archae-bacteria and the like. However, the invention also covers the examination of cell cultures of non-microbial origin, such as cancer cells, immunologically acting cells, epithelial cells, plant cells and the like. The invention therefore also covers applications in the field of functional cell characterisation and the field of toxicological examinations.

The procedure described by the invention is characterised by the fact that it is sensitive, can be standardised and is reproducible. It is generally and uniformly. applicable to the most varying action mechanisms. It is cost-effective and provides quick results.

A further advantage of the procedure described by the invention lies in the possibility of inclusion of mutants of the test germ used, whereby the mutation leads to a sub-expression of a particular target, and in this way regulates the inhibition of this target by a potential inhibitor. With the state of the technology as it exists today, such mutants can easily be created for any required target (Guzman L. M. et al. (1995) Tight regulation, modulation, and high-level expression by vectors containing the arabinose PBAD promoter. J. Bacteriol. 177(14): 4121-30). In this way, the mechanism of inhibiting agents can be determined for such targets for which no reference inhibitors are yet known.

### FIGURES AND EXAMPLES

### Example

Determination of the Minimum Inhibiting Agent Concentration (MIC)

For the production of an overnight culture, 22 ml of Belitsky Minimal Medium (Stuhlke et al. (1993) Temporal activation of beta-glucauase synthesis in Bacillus subtilis is mediated by the GTP pool. J. Gen. Microbiol. 1993 Sep; 139 (pt 9):2041-5) was injected with an aliquot of the test germ Bacillus subtilis 168 from a permanent culture stored at −80° C., and incubated at 37° C. and 200 rpm. The culture, which after 16-18 hrs demonstrated an OD₅₀₀ of 1.0-1.6, was diluted with Belitsky Minimal Medium to an OD₅₀₀ of 0.01 (equivalent to a germ count of approx. 0.8-2×10⁵ germs per ml), and incubated on a 96 micro-titration plate, scale 1:1 with the preparations to be tested placed in the same medium, which were available in serial 1:2 dilutions. The MIC was specified as the lowest concentration of an inhibitor in which no bacterial growth could be observed after 18-24 hrs of incubation at 37° C. table 1 shows the MIC values of the reference substances used for the creation of the reference database.

TABLE 1
Reference substances. MIC values against B. subtilis
168 and the concentrations used.

| reference compounds | MIC [μg/ml] | applied concentration [μg/ml] | | | |
|---|---|---|---|---|---|
| tetracyclin | 16 | 4 | 16 | 64 | |
| chloramphenicol | 4 | 1 | 4 | 16 | |
| methicillin | 0.125 | 0.03 | 0.06 | 0.125 | |
| rifampicin | 0.25 | 0.06 | 0.125 | 0.25 | 1 |
| ciprofloxacin | 0.25 | 0.06 | 0.25 | 0.5 | |
| moxifloxacin | 0.125 | 0.03 | 0.125 | 0.25 | |
| kanamycin | 0.5 | 0.125 | 0.5 | 1 | |
| oxacillin | 0.5 | 0.06 | 0.125 | 0.25 | |
| cefoxitin | 2 | 0.25 | 0.5 | 1 | |
| moxalactam | 4 | 1 | 4 | 8 | |
| erythromycin | 0.5 | 0.125 | 0.5 | 2 | |
| fusidic acid | 0.5 | 0.125 | 0.5 | 2 | |
| nalidixic acid | 32 | 8 | 32 | 128 | |
| novobiocin | 2 | 0.5 | 2 | 8 | |
| trimethoprim | 0.5 | 0.125 | 0.5 | 2 | |
| vancomycin | 0.25 | 0.06 | 0.25 | 0.5 | |
| D-cycloserine | 64 | 4 | 8 | 16 | |
| clindamycin | 2 | 0.5 | 2 | 8 | |
| gentamicin | 0.125 | 0.02 | 0.03 | 0.06 | |
| penicillin G | 4 | 1 | 4 | 16 | |
| neomycin | 0.125 | 0.03 | 0.125 | 0.5 | |
| tobramycin | 0.0625 | 0.02 | 0.06 | 0.125 | |
| mupirocin | 0.0625 | 0.02 | 0.06 | 0.25 | |
| puromycin | 8 | 2 | 8 | 16 | |
| ristocetin | 0.5 | 0.125 | 0.5 | 1 | |
| teichoplanin | 0.125 | 0.03 | 0.125 | 0.25 | |
| spectinomycin | 16 | 4 | 16 | 64 | |
| streptomycin | 128 | 16 | 32 | 64 | |
| clarithromycin | 0.0625 | 0.02 | 0.06 | 0.25 | |
| azithromycin | 1 | 0.25 | 1 | 4 | |
| oxazolidinon BAY | 0.25 | 0.06 | 0.25 | 1 | |
| 11-5845 | | | | | |
| mitomycin C | 0.25 | 0.03 | 0.06 | 0.125 | |
| mersacidin | 16 | 1 | 2 | 4 | |
| ramoplanin | 1 | 0.0625 | 0.125 | 0.25 | |
| actinomycin D | 1 | 0.25 | 1 | 4 | |
| monensin | 4 | 1 | 4 | 16 | |
| gramicidin S | 1 | 0.125 | 0.25 | 0.5 | |
| gramicidin A | 4 | 0.0313 | 0.0625 | 0.125 | |
| lasalocid | 1 | 0.25 | 1 | 4 | |
| nigericin | 1 | 0.03 | 0.06 | 0.125 | |
| nitrofurantoin | 16 | 2 | 4 | 8 | |
| ethidiumbromid | 4 | 1 | 4 | 16 | |
| proflavin | 8 | 2 | 8 | 32 | |
| cerulenin | 16 | 4 | 8 | 16 | 64 |
| doxorubicin | 8 | 1 | 2 | 4 | |
| azaserine | 4 | 1 | 4 | 8 | |
| enniatin | 16 | 4 | 8 | 16 | |
| 5-fluoro-uracile | 0.25 | 0.0625 | 0.25 | 1 | |
| 5-fluor-2-desoxyuridine | 0.25 | 0.0625 | 0.25 | 1 | 4 |
| polymyxin B-sulfate | 16 | 2 | 4 | 8 | 16 |

Cultivation of Cells and Treatment with Reference- and Test Substances

Starting with the overnight culture produced as described above, 50 ml samples of Belitsky Minimal Medium pre-warmed to 37° C. ere each injected with 1 ml of the overnight culture, and incubated at 37° C. and 200 rpm. In the exponential growth phase at OD₅₀₀ 0.25-0.27, the substances were added in the concentrations shown in Table 1, and the mixtures incubated for a further 150 min. As a control, an untreated culture was maintained for each experiment with a single determination. In order to detect internal variances, each preparation was determined five times at every concentration. The concentrations used were selected in advance by means of a growth experiment in such a way that after 150 min acting time, an effect could be seen on the growth speed in comparison to an untreated control culture, although no lytic processes had yet set in—either in the growth curve or under microscopic examination.

Sample Preparation for FT-IR Spectroscopic Investigation

After treatment of the bacteria cells with the reference or test substances for 150 min., 20 ml of each of he cultures was centrifuged in a Heraeus Sepatech Minifuge T at 5.500×g (5,650 rpm) for 10 min. at 16° C. The cell sediments were washed twice with 1 ml of water, the cells being sedimented between the washing steps in an Eppendorf centrifuge at 13,000 rpm for 10 min. The samples were finally placed in water and carefully resuspended, so that after subsequent 30 min. drying of 35 μl of cells at 40-50 mbar at room temperature under P₄O₁₀, homogenous bacterial films formed, whose absorption was in the range of 0.345 to 1.245 absorption units (AU).

The FT-IR spectra of the bacterial cultures treated with the -test substances were recorded using an IFS 28B FT-IR spectrometer (Bruker, Ettlingen) in the absorption mode with a ZnSe sample holder, for 15 sample positions. The spectra were recorded with a DTGS detector and 64 scans in the wavelength range from 4,000-5,000 cm⁻¹. The Fourier transformation was performed with a Blackman-Harris 3-Term apodisation function and a zero-filling factor to produce a spectral resolution of 6 cm⁻¹.

In order to minimise contamination due to water vapour in the room air, the spectrometer was permanently flushed with 500-1,000 1/h of dry air, which was produced with the aid of a Zander air dryer. The water vapour content was measured during the recording of the spectra in the range of 1,837-1,847 cm⁻¹, and measured no more than 0.0003 AU.

Under these conditions, the noise did not exceed 0.0003 AU in the range 2,000-2,100 cm⁻¹,

A quality control check of the FT-IR spectra measured was applied to the spectra, with threshold values for minimum absorption (0.345 AU) and maximum absorption (1.245 AU), which was within the linearity range of the detector.

A background spectrum was recorded before every measurement of a sample, so that compensation could be made for the background.

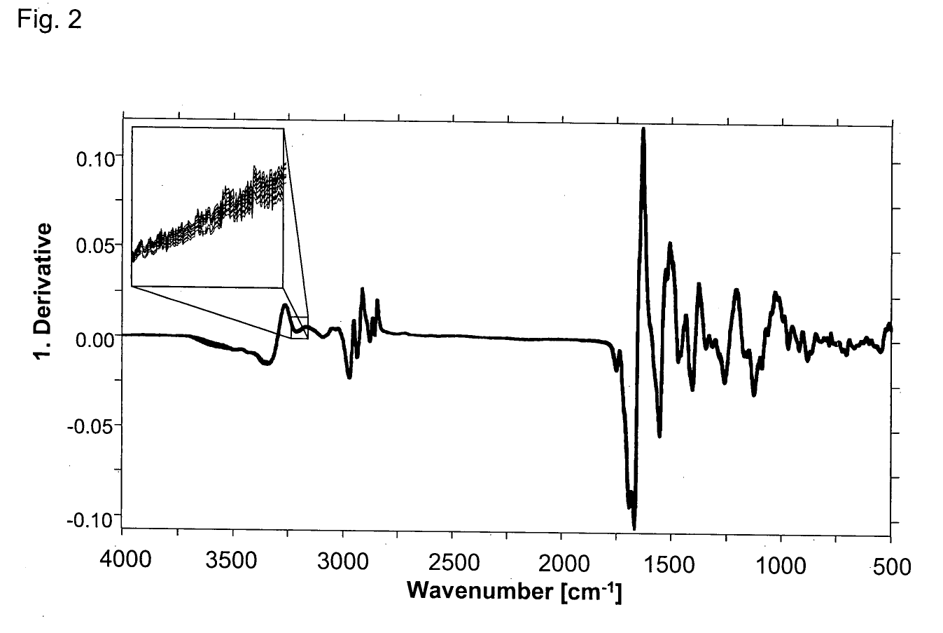

5 separate measurements were carried out for each sample, in order to record variances from measurement to measurement for each sample. The reproducibility of the spectra recordings over a period of six months is shown in FIG. 2. The spectro-photometer was controlled using the Optics user software OPUS 3.0 (Version 970717.0) from Bruker, Ettlingen, Germany.

The mathematical data evaluation procedures described below were applied in order to increase the spectral contrast of the FT-IR spectra after formation of the first derivation using a Savitzky-Golay algorithm (Savitzky A. and Golay M. J. (1964) Smoothing and differentiation of data by simplified least square procedures. Anal. Chem. 36: 1627-1638), taking into account 9 smoothing points and performing a vector normalisation.

Creation of a Mathematical Classification Model:

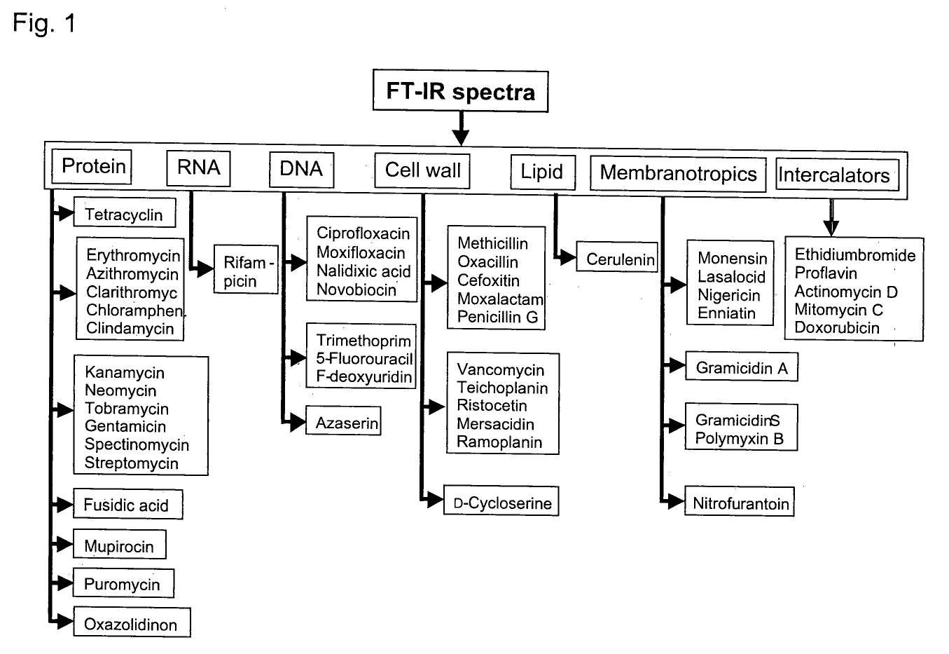

The creation of the mathematical classification model was based on the reference spectra after formation of the 1^{st} derivation. A norming was then carried out for purposes of spectral comparability with regard to the intensities by means of a vector norming (OPUS software manual P. 126, Bruker, Ettlingen). The reference data were then divided into the required number of different action mechanisms, in this example the number being 7 main groups (see FIG. 1). The reference spectra were sorted according to their membership of these 7 main groups. The purpose of this sorting is to use the mathematical procedures to find those wavelengths that are particularly suitable for the classification of the spectral patterns of the individual groups (feature selection). One procedure for wavelength selection used calculates the Euclidian distance of each spectral data point and the centroid (mean point of the class) for every wavelength. The most suitable wavelengths for the classification are those wavelengths whose Euclidian distance within the classes (from the centroid) is as small as possible, but whose separation distance between the different classes is as large as possible. An automated and optimised search for wavelengths that meet these criteria is carried out by means of a genetic algorithm. In this way, the wavelengths can be compiled into a ranking more quickly and efficiently, in the best way possible for the classification. The wavelengths for the classification model with neuronal networks were later selected from this list of wavelengths ranked according to their classification potential.

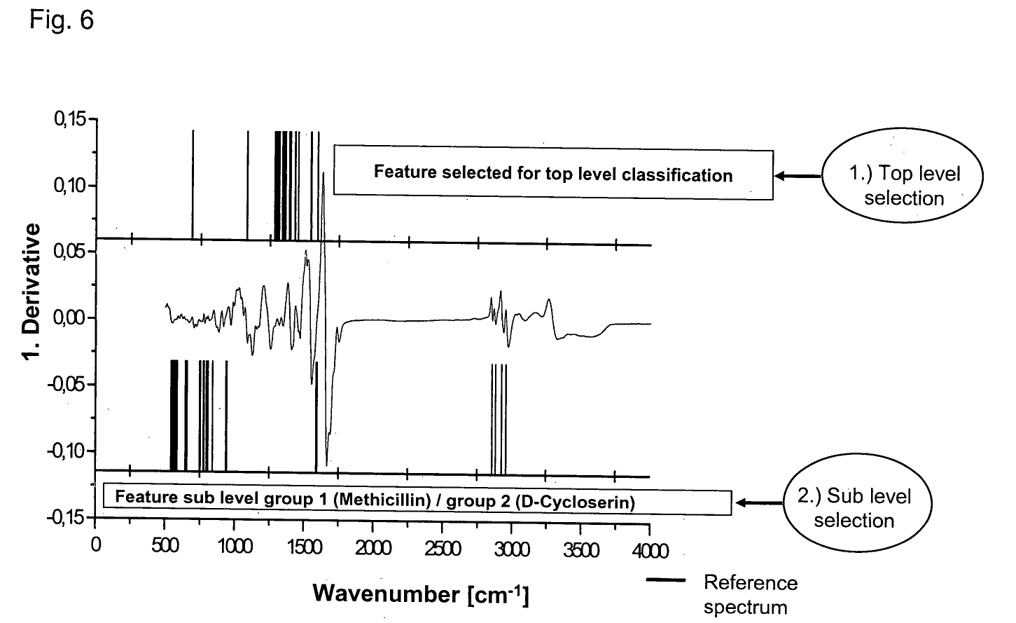

A second approach was based on the calculation of the variances (univariate and covariate) of each data point of the reference spectra within the group, which was then compared with the variance between the groups. An automatic ranking of the wavelengths was then carried out, in which the variance within the group is as small as possible, and the variance between the different groups as large as possible. The best 97 wavelengths from this ranking were used as input neurons for a neuronal network. The wavelength selection using this procedure is shown in FIG. 6.

The classification model used was a three-layer feed-forward network with 07 input neurons, 22 hidden neurons and 7 output neurons, The resilient back-propagation algorithm (RProp) was used as the learning algorithm. The output activation was set between 0 and 1.

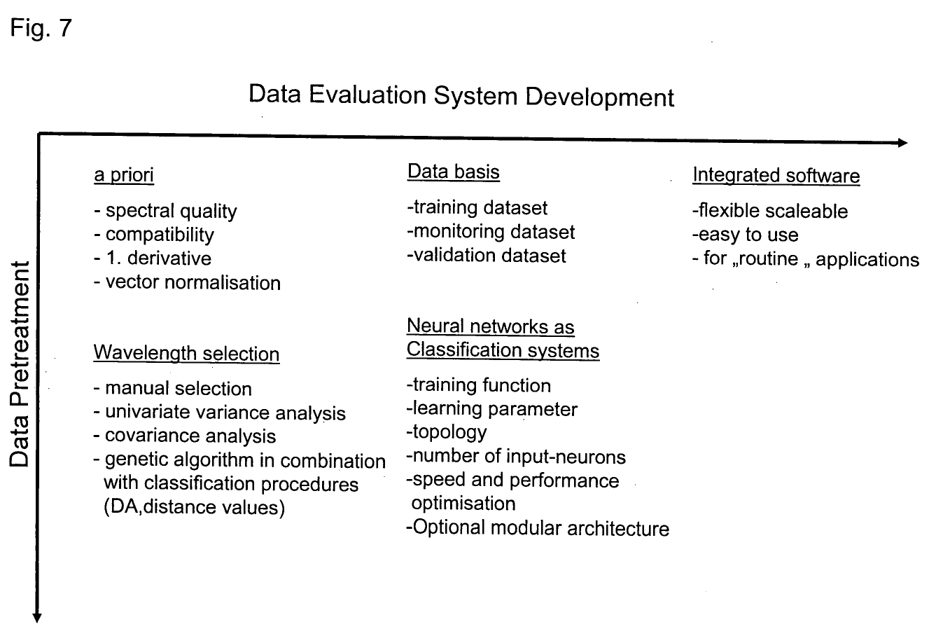

FIG. 7 shows the data processing concept

Classification of a Substance X with Unknown Mode of Action Mechanism:

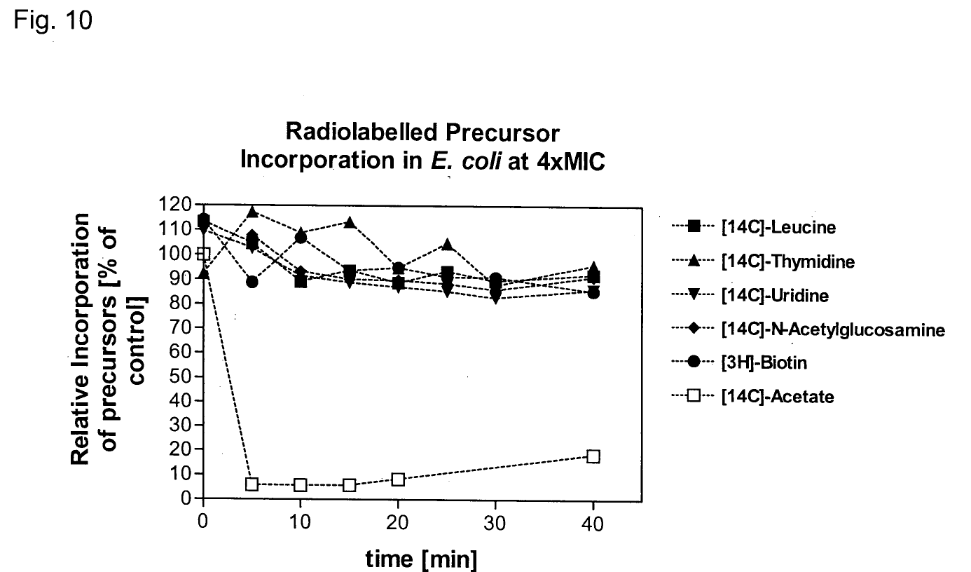

For the external validation of the procedure described, the bacterial cells were treated with the antibacterial acting substance X (MIC 2 μg/ml) and determined five times at the concentrations of 1, 2 and 4 μg/ml. The performance of the classification procedure, under treatment with 2 and 4 μg/ml, in all cases produced a clear allocation of the spectra into the class of samples treated with Cerulenin. Cerulenin is an inhibitor of the fatty acid biosynthesis metabolism, which gives rise to the suspicion that substance X has an action mechanism similar to Cerulenin. In fact, FIG. 10 shows that substance X selectively inhibits the de novo incorporation of [¹⁴C]-acetate in CHCl₃/MeOH extractable phospholipids. The evaluation of the spectra of the bacteria treated with only 1 μg/ml of substance X produced no such allocation, which possibly because of the low dose could be due to the only very minor changes in the growth curve and the FT-IR spectrum in comparison to the untreated control cultures.

The figures show

FIG. 1 Structure of the reference database on the basis of the action mechanisms of known antibiotics

FIG. 2 Reproducibility of the spectral measurements

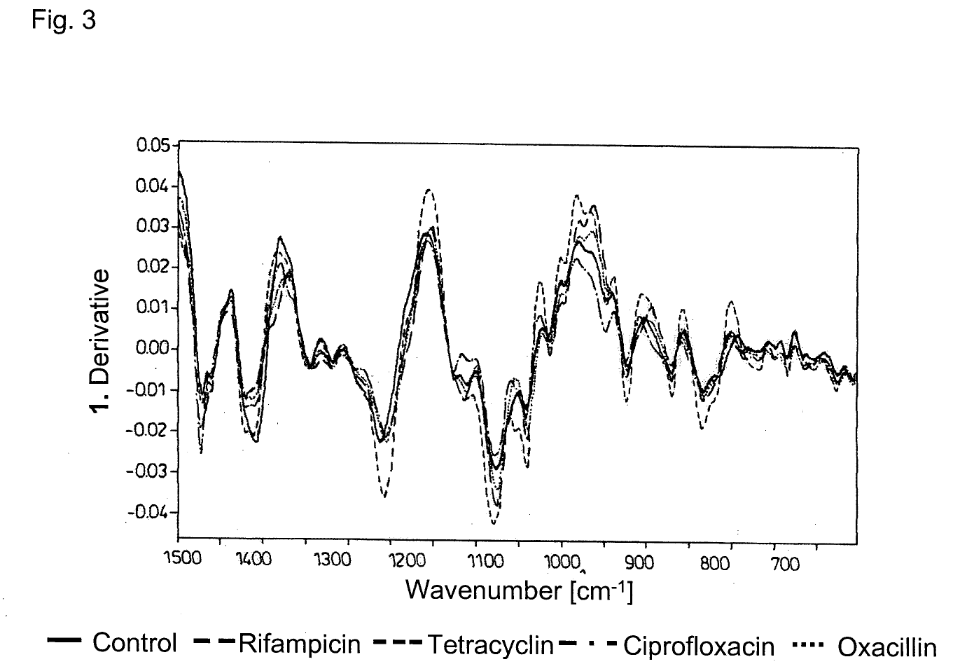

FIG. 3 Differentiation of antibiotics classes

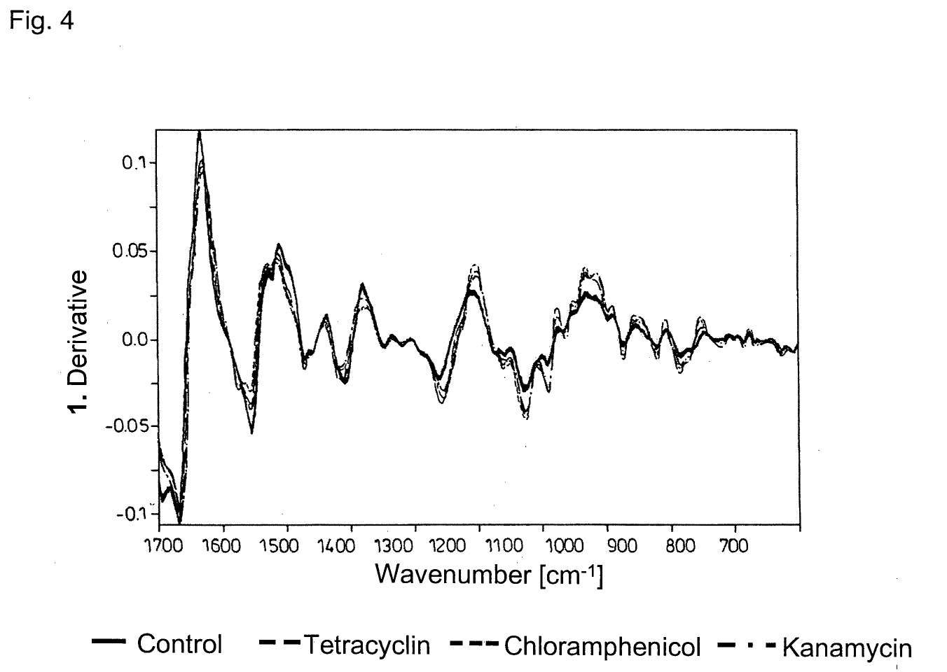

FIG. 4 Spectra of protein biosynthesis inhibitors

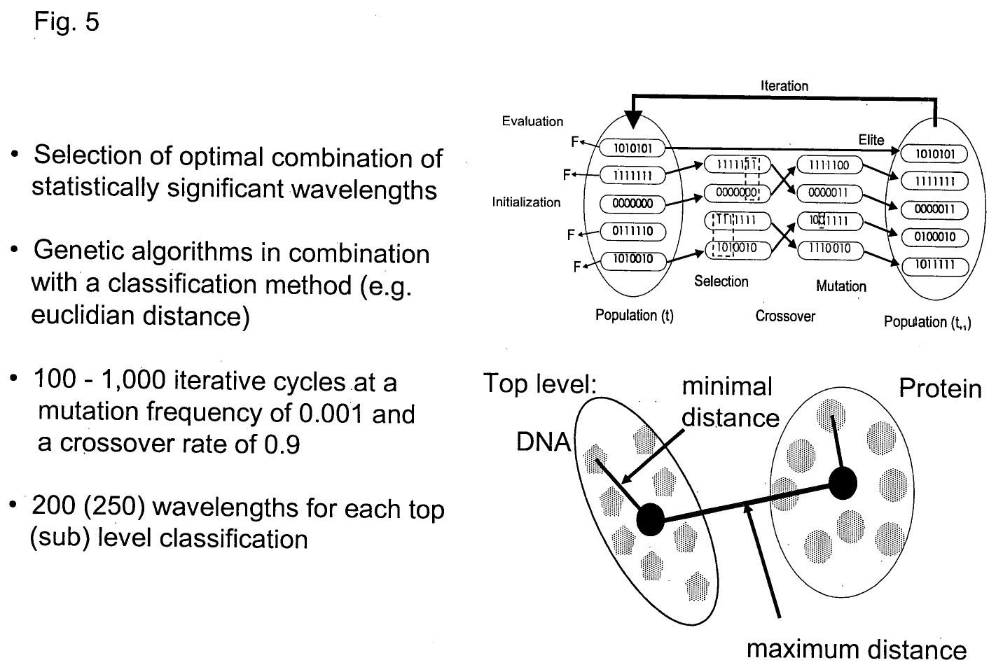

FIG. 5 Wavelength selection procedures

FIG. 6 Hierarchical allocation of action mechanisms

FIG. 7 Data processing concept

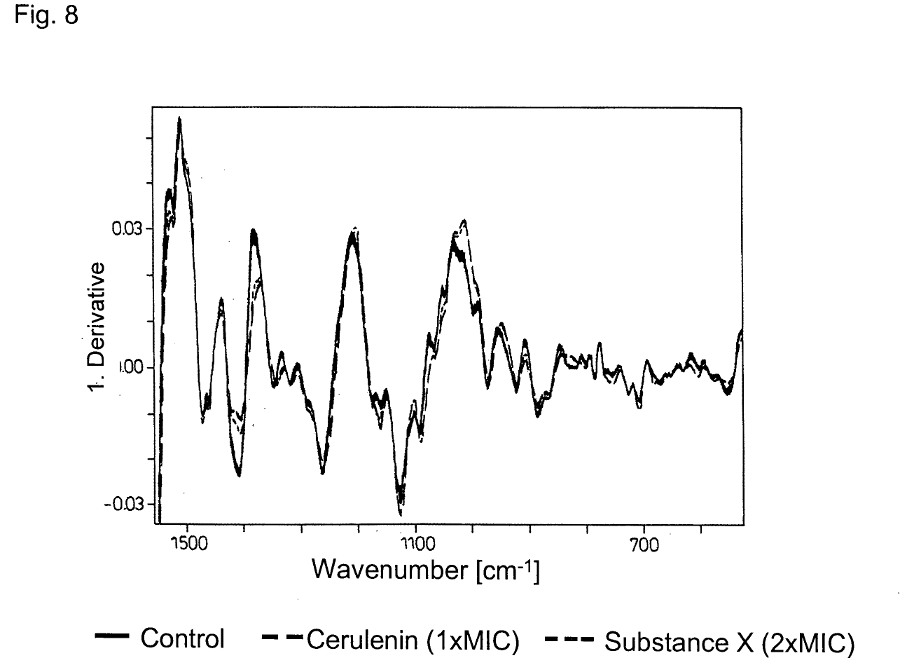

FIG. 8 Example action mechanism of substance X

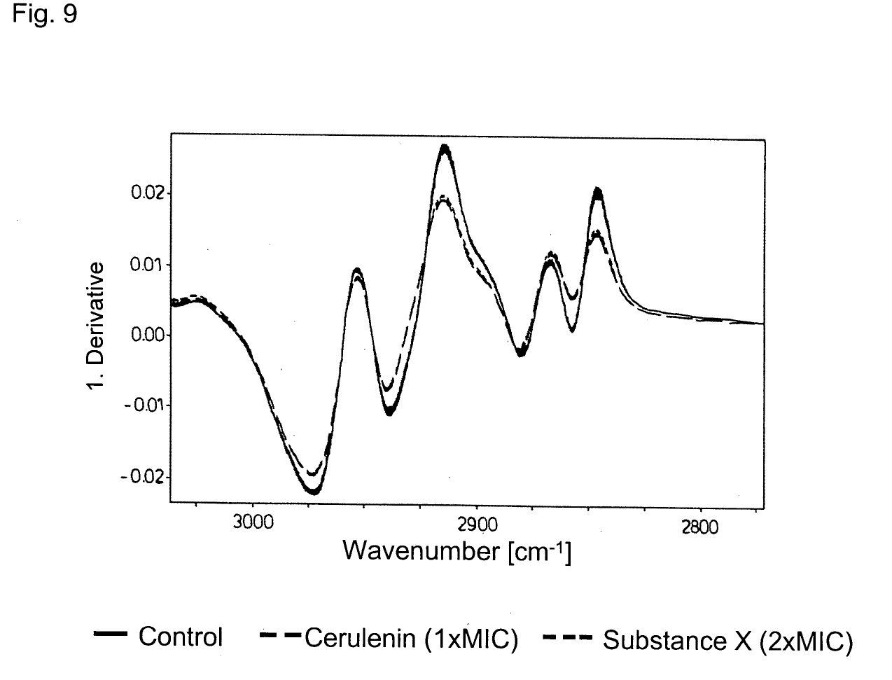

FIG. 9 Evaluation of the spectrum of substance X in a 1^{st} wavelength range

FIG. 10 Evaluation of the spectrum of substance X in a 2^{nd} wavelength range

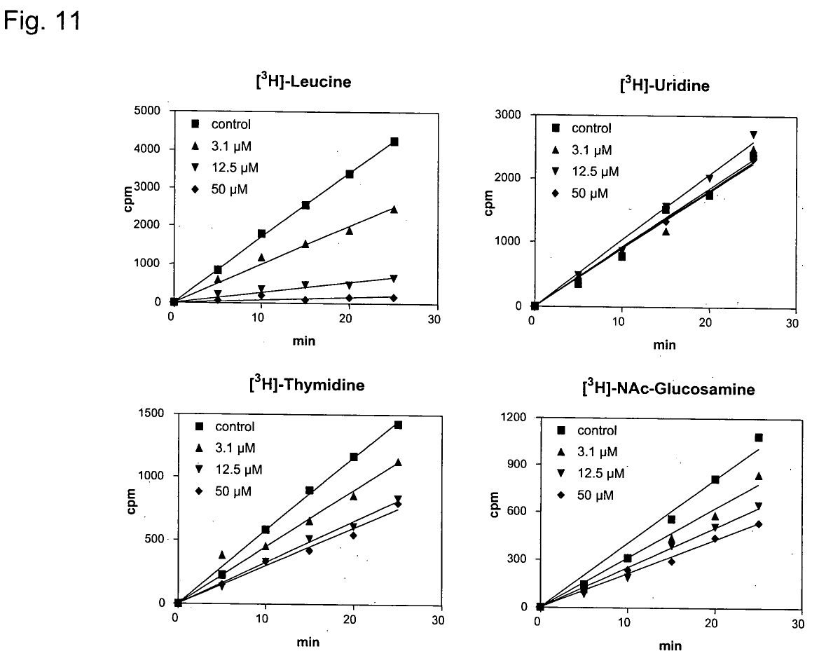

FIG. 11 Example action mechanism of substance Y

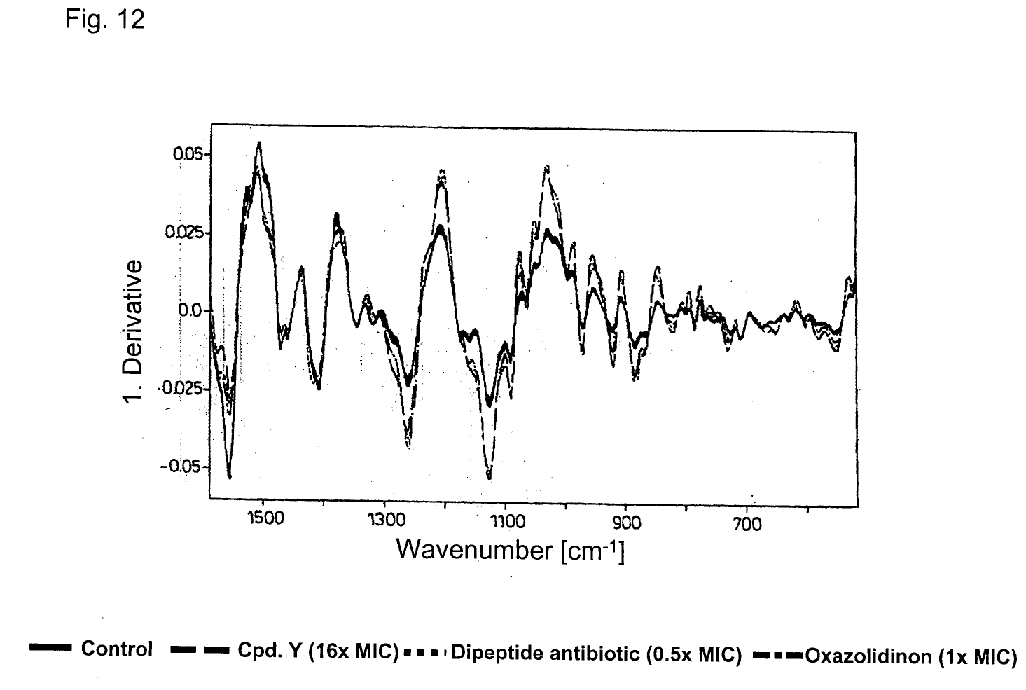

FIG. 12 Evaluation of the spectrum of substance Y

FIG. 1 shows the arrangement of the classification system used for the example in the form of hierarchical neuronal networks, together with the allocation of the reference antibiotics. In the first classification step, the 7 main classes of inhibitors (protein biosynthesis inhibitors, RNA biosynthesis inhibitors, DNA biosynthesis inhibitors, cell wall biosynthesis inhibitors, lipid biosynthesis inhibitors, membrano-tropic substances and intercalators) are separated from each other. In a second step, sub-groups are then defined (e.g. DNA biosynthesis inhibitors with the 3 sub-groups 1. Ciprofloxacin-like substances, 2. Trimethoprim-like substances, 3. Azaserin-like substances. This division into sub-groups can in principle be continued and extended. The allocations made are directly confirmable for the specialist in the field, and can be derived from the relevant reference works (e.g. Graefe U. (1992) Biochemie der Antibiotika, pp. 15-39, Spektrum Akademischer Verlag Heidelberg, Berlin, N.Y.).

FIG. 2 shows the superimposition of the 1^{st} derivation of 25 randomly selected spectra of the microorganism Bacillus subtilis strain 168 without the addition of an inhibiting agent. The spectra were recorded over a period of 6 months. All 25 spectra are practically identical, and show only negligible variance. This demonstrates the good reproducibility of the recording of spectra of microbial cultures. This reproducibility is an important requirement for the success of the procedure described by the invention.

FIG. 3 shows the 1^{st} derivative spectra of 25 control spectra, taken in independent experiments, of a bacterial culture of Bacillus subtilis strain 168 without treatment with a test substance, and, superimposed 5 times, the 1^{st} derivative spectra of spectra of bacterial cultures of the, same strain, that have been treated with the different antibiotics Rifampicin, Tetracyclin, Ciprofloxacin and Oxacillin. as shown in FIG. 1, the different antibiotics are allocated different action mechanisms. The spectra of the bacterial cultures treated with the different antibiotics therefore vary accordingly. The acting time was in each case 150 min., the concentration was 4× the minimum inhibiting agent concentration (MIC), or 0.25× MIC in the case of Tetracyclin. The MIC values of the antibiotics are 0.25 μg/ml for Rifampicin, 16 μg/ml for Tetracyclin, 0.25 μg/ml for Ciprofloxacin and 0.5 μg/ml for Oxacillin.

FIG. 4 shows the 1^{st} derivative spectra of 25 control spectra of a bacterial culture without treatment with a test substance, and, superimposed 5 times, the 1^{st} derivative of spectra of bacterial cultures treated with the different antibiotics Tetracyclin (4 μg/ml), Chloramphenicol (4 μg/ml) and Kanamycin (4 μg/ml). The treatment time of the bacterial cultures was in each case 150 min. All three antibiotics tested here are protein biosynthesis inhibitors. The 1^{st} derivative of the spectra of the spectra treated with these different protein biosynthesis inhibitors demonstrate good correlation amongst each other, and significant differences to the 1^{st} derivative of the control spectra.

FIG. 5 explains an example of a procedure for wavelength selection. In this procedure, the Euclidian distance of every spectral data point is calculated, and the centroid (mean point of the class) for every wavelength calculated. The most suitable wavelengths for the classification are those wavelengths whose Euclidian distance within the classes (from the centroid) is as small as possible, but whose separation distance between the different classes is as large as possible. An automated and optimised search for wavelengths that meet these criteria is carried out by means of a genetic algorithm. In this way, the wavelengths can be compiled into a ranking more quickly and efficiently, in the best way possible for the classification. The wavelengths for a classification model (e.g. neuronal networks), ranked according to their classification potential, will later be selected from this list of wavelengths.

FIG. 6 shows the hierarchical allocation of the action mechanism. The black bars represent those wavelength ranges used for the classification of the antibiotics according to their action mechanisms. The upper part of the figure shows the spectral ranges that demonstrate a particularly high significance for the separation of the 7 main groups (inhibitors of protein, RNA, DNA, lipid and cell wall synthesis, together with membrano-trophic substances and intercalators); the lower part of the figure shows in contrast the spectral ranges used for the classification of the antibiotics into different sub-groups within the main groups by means of the example of the separation of β-Lactames and D-cycloserin within the main group of cell wall synthesis inhibitors.

FIG. 7 shows the data processing concept.

FIG. 8 shows the action mechanism of a substance X. Substance X selectively inhibits the de novo incorporation of [¹⁴C]-acetates in CHCl₃/MeOH extractable phospholipids.

FIG. 9 shows the 1^{st} derivation of 25 control spectra of a bacterial culture without treatment with a test substance, and, superimposed 5 times, the 1^{st} derivative of spectra of bacterial cultures treated with the Cerulenin (1× MIC; 16 μg/ml) and substance X (2× MIC; 2 μg/ml). As can be seen from FIG. 1, Cerulenin is a lipid synthesis inhibitor. The similarity of the FT-IR pattern indicates that the unknown test substance X also acts as an inhibitor of lipid synthesis. FIG. 10 shows the same spectra as FIG. 9, but in a different wavelength range. This spectral range is dominated by vibration transitions of the fatty acid molecules. In this spectral range, the differences between the reference spectra and the test spectra with the lipid-synthesis-inhibiting test substances are particularly significant.

FIG. 11 shows the action mechanism of a substance Y. Substance Y selectively inhibits the de novo incorporation of [³H]-leucin in perchloric acid precipitable material.

FIG. 12 shows the 1^{st} derivation of a control spectrum of a bacterial culture without treatment with a test substance, and, superimposed, the 1^{st} derivation of spectra of bacterial cultures treated with a dipeptide antibiotic (0.5× MIC; 0.5 mg/L), an oxazolidinon (1× MIC; 2 mg/L) and the substance Y (16× MIC; 3 mg/L). As can be seen from FIG. 1, the oxazolidinon is a protein biosynthesis inhibitor, while the same applies for the dipeptide antibiotic. The similarity of the IR pattern indicates that the unknown test substance Y also acts as an inhibitor of protein biosynthesis.

1-26. (canceled)

27. Process for the identification and/or characterisation of the action mechanism of an antimicrobial substance comprising the following steps:
a) Compilation of reference spectra by means of the treatment of certain microbial cultures with test substances whose action mechanism is known, and recording of at least one spectrum from the group of IR, FT-IR, Raman and FT-Raman spectra.
b) In each case, selection of at least one wavelength range of the same or similar structure to differentiate between the classes belonging to the corresponding action mechanism, and allocation of the reference spectra into the classes in the reference database, whereby the reference spectra allocated to a class demonstrate an identical or similar structure in the selected wavelength range, which differs significantly from the structure of the reference spectra of other classes in the selected wavelength range.
c) Treatment of a microbial culture with the substance to be tested.
d) Recording of at least one spectrum (test spectrum) from the group of IR, FT-IR, Raman and FT-Raman spectra.
e) Comparison of the test spectrum/spectra from d) with one or more reference spectra in the reference database.
f) Allocation of the test spectra to one, two or more classes of reference spectra in the reference database and identification or characterisation of the action mechanism.

28. Process according to claim 27, wherein the comparison e) is carried out with the aid of mathematical methods of pattern recognition.

29. Process according to claim 27, wherein that the spectra referred to in d) are processed in such a way as to enable the automatic recognition of the characteristic spectral changes and patterns.

30. Process according to claim 27, wherein the classification is carried out by means of pattern recognition that can separate two or more classes simultaneously.

31. Process according to claim 27, wherein the information of a spectral pattern characteristic of one of the classes is stored in a classification model or in the form of weights of synthetic neuronal networks.

32. Process according to claim 27, wherein the comparison of the test spectra with the reference spectra is carried out by means of the classification model.

33. Process according to claim 27, wherein the microbial culture is a pure culture.

34. Process according to claim 27, wherein the action mechanism comprises inhibitors of protein biosynthesis, the RNA or DNA metabolism, the cell wall or lipid metabolism, membrano-trophic substances or DNA intercalators.

35. Process according to claim 27, wherein the defined mutants of the microbial germ are also used for the creation of the reference database, whereby the mutation of the target gene concerned regulates the interaction of the gene product with a hypothetical reference substance.

36. Process according to claim 27, wherein the mutants are those with reduced or increased production of a selected target gene, or those with reduced or increased biological activity because of point mutations and/or deletions.

37. Process according to claim 27, wherein the selection of the wavelength ranges used for the differentiation of the classes (wavelength selection) is made by means of multi-variate statistical procedures, together with an optimisation process such as genetic algorithms.

38. Process according to claim 37, wherein the multi-variate statistical procedures are selected from variance analysis, co-variance analysis, factor analysis, statistical distance dimensions, the Euclidian distance or the Mahalanobis distance, or a combination of these methods.

39. Process according to claim 27, wherein prior to the wavelength selection, preliminary processing of the reference spectra is carried out in order to increase the spectral contrast by means of the formation of derivations, deconvolution, filtering, noise suppression or data reduction by wavelet transformation or factor analysis.

40. Process according to claim 27, wherein the allocation of the reference spectra into the different classes is carried out by means of mathematical classification methods of pattern recognition, a general linear model, synthetic neuronal networks, methods of case-based classification, vector optimisation or machine learning, genetic algorithms or methods of evolutionary programming.

41. Process according to claim 27, wherein the allocation of the reference spectra into the different classes is carried out by means of mathematical classification methods.

42. Process according to claim 41, wherein the mathematical classification methods are selected from multi-variate, statistical processes of pattern recognition, neuronal networks, methods of case-based classification and machine learning, genetic algorithms and methods of evolutionary programming.

43. Process according to claim 41, wherein several synthetic neuronal networks and classification methods are used.

44. Process according to claim 43, wherein several synthetic neuronal networks are used as a feed-forward network with three layers and a gradient decline method as the learning algorithm.

45. Process according to claim 43, wherein the classification system has a tree structure, in which classification tasks are broken down into partial tasks, and the individual classification systems in a unit are combined to form a hierarchical classification system, in which all stages of the hierarchy are processed automatically during the course of the evaluation.

46. Process according to claim 45, wherein the individual classification systems comprise neuronal networks optimised for special tasks.

47. Process according to claim 27, wherein the allocation of a test spectrum to one, two or more classes is carried out by means of mathematical classification methods.

48. Process according to claim 47, wherein the classification method is selected from multi-variate, statistical processes of pattern recognition, neuronal networks, methods of case-based classification and machine learning, genetic algorithms and methods of evolutionary programming.

49. Process according to claim 27, wherein the recording of IR spectra is performed in the spectral range of 500-4,000 cm⁻¹ and/or 4,000-10,000 cm⁻¹.

50. Process according to claim 27, wherein the test substance is an inhibiting agent.

51. Process according to claim 27, wherein the concentration of inhibiting agent with which the bacterial culture is treated lies in the range of 0.1× to 20× the minimum inhibiting agent concentration (MIC) for the test substance.

52. Process according to claim 27, wherein test spectra of a microbial culture are recorded which have in all cases been treated with the same inhibiting agent, although in different concentrations.

53. Process according to claim 27, wherein measurement is carried out in cuvettes, throughflow cuvettes and micro-cuvettes, which are measured in transmission, absorption and reflection, and are suitable for automated measurements/throughflow measurements and high-throughput screening.

54. Process according to claim 27, wherein FT-IR, IR, Raman and FT-Raman measurements can be measured directly in sample preparation liquids and vessels.

55. Process according to claim 27, wherein pro- or eucaryontic cells are used as microbial cell cultures.

56. Process according to claim 55, wherein the cell culture is selected from bacteria, moulds, yeasts an archae-bacteria.

57. Process according to claim 27, wherein cell cultures of non-microbial origin can also be examined.

58. Process according to claim 56, wherein the cell cultures are from the group of cancer cells, immunologically acting cells, epithelial cells and plant cells.
